(19) United States
(12) Patent Application Publication
(10) Pub. No.: US 2024/0021013 A1
(43) Pub. Date: Jan. 18, 2024

(54) AUTOMATED ESTIMATION OF BLOOD ALCOHOL CONCENTRATION

(71) Applicant: Bacpoint, Inc., Porter Ranch, CA (US)

(72) Inventors: Victoria Nadershahi, Los Angeles, CA (US); Arman Nadershahi, Los Angeles, CA (US)

(73) Assignee: Bacpoint, Inc., Los Angeles, CA (US)

(21) Appl. No.: 18/222,351

(22) Filed: Jul. 14, 2023

Related U.S. Application Data
(60) Provisional application No. 63/389,361, filed on Jul. 14, 2022.

(51) Int. Cl. G06V 40/16; G01N 33/497; G06V 10/82; G06V 10/774; G06V 40/20; G06V 20/40

(52) U.S. Cl. CPC G06V 40/166; G01N 33/4972; G06V 10/82; G06V 10/774; G06V 40/20; G06V 20/40

(57) ABSTRACT

This disclosure relates to technology that enables a computing system to provide users health information related to their alcohol consumption. Additionally, the present disclosure is directed to a software tool that enables a computing device to estimate a user's blood alcohol concentration based on various combinations of the user's physiological and behavioral information and photographs of the user's face. The disclosure further relates to the use of machine learning to train models to estimate a user's blood alcohol concentration using physiological information and alcohol consumption data obtained from users of the technology.

### CROSS-REFERENCE TO RELATED APPLICATION

This application is related, and claims priority, to U.S. Provisional Patent Application No. 63/389,361 filed Jul. 14, 2022, the entire contents of which are incorporated herein by reference.

### BACKGROUND

### Technical Field

The present disclosure is directed to a software tool that enables a computing device to provide users with health information related to their alcohol consumption. Additionally, the present disclosure is directed to a software tool that enables a computing device to estimate a user's blood alcohol concentration based on the user's physiological and behavioral information.

### Description of Related Art

Alcohol consumption plays an important role in social, religious, and ritualistic settings. Once ingested, metabolism of ethanol begins immediately, eventually yielding roughly 7 kcal per gram. However, unlike other macronutrients such as carbohydrates and lipids that are processed under the regulation of hormones such as leptin, ghrelin, and insulin, ethanol remains in the body's water reservoir until it is processed by the liver and ultimately excreted. Ethanol's journey from lips to liver consists of its passage across numerous different tissues and membranes, through the bloodstream, and involves the interaction of multiple enzymatic reactions. Thus, this process of absorption is subject to influence from a myriad of factors including age, sex, genetic profile, BMI, fasting vs fed state, and active substance interactions. Blood Alcohol Concentration (BAC) refers to the percentage of alcohol (ethyl alcohol or ethanol) circulating in an individual's blood stream. While direct measurement of BAC from blood samples and indirect measurements from breathalyzers are possible, accurate and convenient estimation of BAC remains challenging.

Furthermore, traditional methods for providing health advice and monitoring alcohol consumption are generally static, impersonal, and lack real-time feedback. These methods often fail to engage users or provide personalized, actionable advice.

### SUMMARY

This disclosure relates to technology that enables a computing system to estimate a user's Blood Alcohol Concentration (BAC) based on information obtained from the user through a computing device such as a smartphone. In some exemplary embodiments, the information obtained from a user may comprise, for example: amount of alcohol ingestion; time of alcohol consumption; type of alcohol consumed; time, type, and amount of food ingested; time and amount of water ingested; age; biological sex; height; weight; ethnicity; body mass index (BMI); and total body water. The obtained data may be used by data storage and computing devices to compute various new information, which may then be presented to a user of the computer system.

For instance, in one implementation, the disclosed software technology may cause a computing device to engage in the following operations: (1) receiving data about a plurality of physiological and behavioral parameters of a user; (2) compiling a dataset based on the received data; and (3) generating a health-related recommendation based on the compiled data set and algorithmic operations performed on the data. However, it should be understood that the disclosed software technology for generating health information related to blood alcohol concentration may cause a computing system to perform various other operations as well.

In another aspect, disclosed herein is a computing system that comprises at least one processor, a non-transitory computer-readable medium, and program instructions stored on the non-transitory computer-readable medium that are executable by the at least one processor to cause the computing system to carry out the operations disclosed herein, including but not limited to the operations of the foregoing method.

In yet another aspect, disclosed herein is a non-transitory computer-readable medium comprising program instructions that are executable to cause a computing system to carry out the operations disclosed herein, including but not limited to the operations of the foregoing method.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or to provide more effective illustration. Some embodiments may be practiced with additional components or steps and/or without all of the components or steps that are illustrated. When the same numeral appears in different drawings, it refers to the same or like components or steps.

FIG. 1 is a block diagram illustrating an example system for machine learning model training and deployment for the estimation of blood alcohol concentration.

FIG. 2 is a flow diagram illustrating an example method or system for computing and delivering health information to a user related to the user's consumption of alcohol.

FIG. 3 is a flow diagram illustrating an example method or system for computing and delivering health information to a user related to the user's consumption of alcohol which comprises using the user's images.

FIG. 4 is a flow diagram illustrating an example method or system for computing and delivering health information to a user related to the user's consumption of alcohol which comprises using the user's images and additional information related to the user's consumption of alcohol.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

The present invention provides methods and systems for collecting health data from a user's device, computing additional health information from the collected data, and providing the additional data to the user's device. In an exemplary embodiment, the collected health data may comprise physiological data about the user, data about alcohol consumption such as amount, time, and type of alcohol consumed, water consumption, exercise, food consumption, and images of the user before, after, and/or during the drinking period. Exemplary embodiments of the present invention may comprise assembling a dataset on a server using the collected data and additional data, and then using machine learning algorithms to develop models that can be used to predict the blood alcohol concentration of users when the server receives user data. In some exemplary embodiments of the present invention, the computed health data is an estimate of the user's blood alcohol concentration.

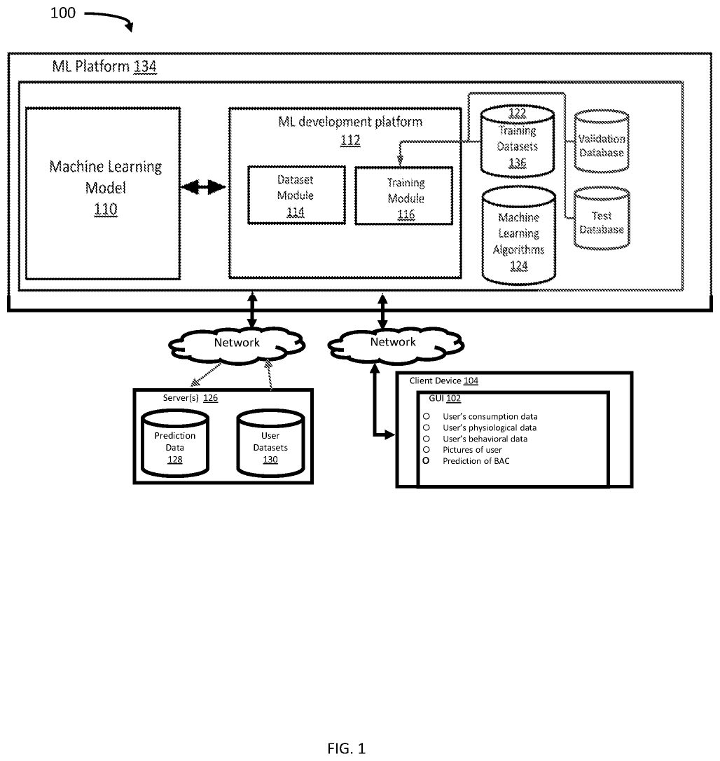

To further describe the present technology, examples are now provided with reference to the figures. FIG. 1 is a block diagram illustrating an example of a system 100 on which the present technology may be executed. As illustrated, the system 100 may include a machine learning (ML) platform 134 that includes components such as a training dataset, validation dataset, and a combination of regression-based algorithms to train a machine learning model 110 to generate predictions of a user's blood alcohol concentration. Components of the ML platform 134 may be hosted on computing resources located, for example, in a service provider environment (e.g., a “cloud” environment), or as other examples, in a private network or colocation network environment.

A client device 104 may comprise various information used by the ML platform 134. An exemplary client device comprises a smartphone comprising a touchscreen and a camera. A graphical user interface (GUI) 102 may allow the user to input information that may be used by the ML platform 134 and allows the user to receive information produced by the ML platform 134. In one example, the GUI 102 can be implemented using a web application configured to execute client-side logic in a browser application on a client device 104. In another example, the GUI 102 can be implemented using an application hosted on a client device 104 that connects to the ML platform 134 over one or more networks. In an exemplary embodiment, the GUI 102 may allow a user to input data such as the user's alcohol, water, or food consumption in addition to using the health metrics that the smartphone tracks by default, such as fitness information in the form of calories burned, carbohydrates burned, and other variables. In an exemplary embodiment, the GUI 102 may allow a user to input data such as the user's physiological data, which may comprise weight, height, age, ethnicity, or sex. In an exemplary embodiment, the GUI 102 may allow a user to input data such as the user's exercise information, sleep information, or movement. The GUI 102 may also allow for the retrieval of a smartphone's collected data, or a wearable device's collected data that the user has granted access to. In an exemplary embodiment, the GUI 102 may allow a user to input data such as pictures of the user during, before, or after a drinking episode. In some exemplary embodiments, the client device 104 automatically collects various user data and sends it to the ML Platform 134 via a network. In an exemplary embodiment, the ML platform 134 can use datasets obtained through trials and published data to train a machine learning model, and then use the user data to further refine the model's prediction accuracy and precision. In an exemplary embodiment, the machine learning model 110 can use the user's data to compute and provide information to the user related to the user's alcohol consumption.

The ML development platform 112 may include tools for analyzing user data sets 130, training machine learning models using various machine learning algorithms 124, comparing the performance of machine learning models of various types and versions, a machine learning model 110 to generate predictions of a blood alcohol concentration, among other tools. The ML development platform 112 may contain modules configured to provide the tools for building, training, analyzing, and deploying machine learning models. The modules can include a dataset module 114, a training module 116, and other modules.

The dataset module 114 can be configured to obtain user datasets 130 for use in training a machine learning model 110. A user dataset 130 can be hosted on computing resources (e.g., server(s) 126) located in a service provider environment, located in a user's private network, or located in other locations, as can be appreciated. A user dataset can comprise structured data (e.g., categorized and/or formatted data) having data elements which can be analyzed to identify associations between the data elements. Illustratively, data contained in a user dataset 130 may include data such as weight, height, age, ethnicity, or sex. The user dataset 130 may also include blood alcohol information from users, such as readings from breathalyzers. The user dataset 130 may also include genetic information from users.

Prediction data 128 generated by the machine learning model 110 can be sent to a client device 104 to allow a user to access the prediction data.

In one example, the dataset module 114 may be configured to retrieve various data for the user datasets 130 and the data can be used to train a machine learning model 110. A user dataset 130 can be stored on the ML platform 134 in a sync data store 122 as training datasets 136. In one example, the dataset module 114 synchronizes user datasets 130 stored on server 126 with training datasets 136 stored in the sync data store 122.

The dataset module 114, in one example, can be configured to analyze data elements contained in a user dataset 130 to determine whether the data elements correlate to a target metric such as blood alcohol concentration. The dataset module 114 can be configured to select the data elements that correlate to the target metric and include the data elements in a training dataset 136. In another example, dataset module 114 can be configured to analyze a training dataset 136 to identify data elements that do not correlate to a target metric and remove the data elements from the training dataset 136. Also, in some examples, the dataset module 114 can be configured to perform actions that prepare a training dataset 136 to be used to train a machine learning model 110, such as, but not limited to, replacing null values with a placeholder value, normalizing data, standardizing data, aggregating data, detecting and removing outlier data, dividing datasets into training, cross-validation, and testing datasets, as well as other actions. For example, the dataset module 114 can be configured to analyze a training dataset 136 based on an action to be performed (e.g., normalizing, aggregating, dividing, etc.) and perform the action based on the analysis. In another example, a user can use the GUI 102 to evaluate a training dataset 136 and use dataset preprocessing tools (e.g., normalization tools, aggregation tools, dataset dividing tools, etc.) provided by the dataset module 114 to prepare the training dataset 136 for training a machine learning model 110.

In one example, the dataset module 114 can be configured to analyze a data element in a training dataset 136 to determine usefulness of a data element to predict a target metric and remove the data element from the training dataset 136 when the data element is determined to be not useful in predicting the target metric. For example, a data element can be analyzed to determine whether an occurrence of unique values (high cardinality, e.g., where each row contains a unique value for the column), null values (e.g., a column in a dataset contains a high number of null values), single value (e.g., a column in a dataset contains the same value in each row), and/or other values represented by the data element warrant removing the data element from a training dataset 136, and removing the data element from the training dataset 136 when the determination warrants removal of the data element.

The ML development platform 112 can be configured to generate a prediction score for a machine learning model 110 which can be used to evaluate performance of the machine learning model 110. Various methods can be used to generate a prediction score, including: Fi score (also F-score or F-measure), area under curve (AUC) metric, mean squared error, receiver operating characteristic (ROC) curve metric, relevance and ranking (in information retrieval), as well as other scoring methods.

The various processes and/or other functionality contained within the system 100 may be executed on one or more processors that are in communication with one or more memory modules. The system 100 may include a number of computing devices that are arranged, for example, in one or more server banks or computer banks or other arrangements. The computing devices may support a computing environment using hypervisors, virtual machine monitors (VMMs) and other virtualization software. The term “data store” may refer to any device or combination of devices capable of storing, accessing, organizing and/or retrieving data, which may include any combination and number of data servers, relational databases, object-oriented databases, cluster storage systems, data storage devices, data warehouses, flat files and data storage configuration in any centralized, distributed, or clustered environment. The storage system components of the data store may include storage systems such as a SAN (Storage Area Network), cloud storage network, volatile or non-volatile RAM, optical media, or hard-drive type media. The data store may be representative of a plurality of data stores as can be appreciated.

API calls, procedure calls or other network commands that may be made in relation to the modules and services included in the system 100 may be implemented according to different technologies, including, but not limited to, Representational State Transfer (REST) technology or Simple Object Access Protocol (SOAP) technology or GraphQL. REST is an architectural style for distributed hypermedia systems. A RESTful API (which may also be referred to as a RESTful web service) is a web service API implemented using HTTP and REST technology. SOAP is a protocol for exchanging information in the context of Web-based services.

A network can be used for communications between components of the system 100. The network may include any useful computing network, including an intranet, the Internet, a local area network, a wide area network, a wireless data network, or any other such network or combination thereof. Components utilized for the network may depend at least in part upon the type of network and/or environment selected. Communication over the network may be enabled by wired or wireless connections and combinations thereof.

FIG. 1 illustrates that certain processing modules may be discussed in connection with this technology and these processing modules may be implemented as computing services. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, virtualized service environment, grid or cluster computing system. An API may be provided for each module to enable a second module to send requests to and receive output from the first module. Such APIs may also allow third parties to interface with the module and make requests and receive output from the modules. While FIG. 1 illustrates an example of a system that may implement the techniques above, many other similar or different environments are possible. The example environment discussed and illustrated above is merely representative and not limiting.

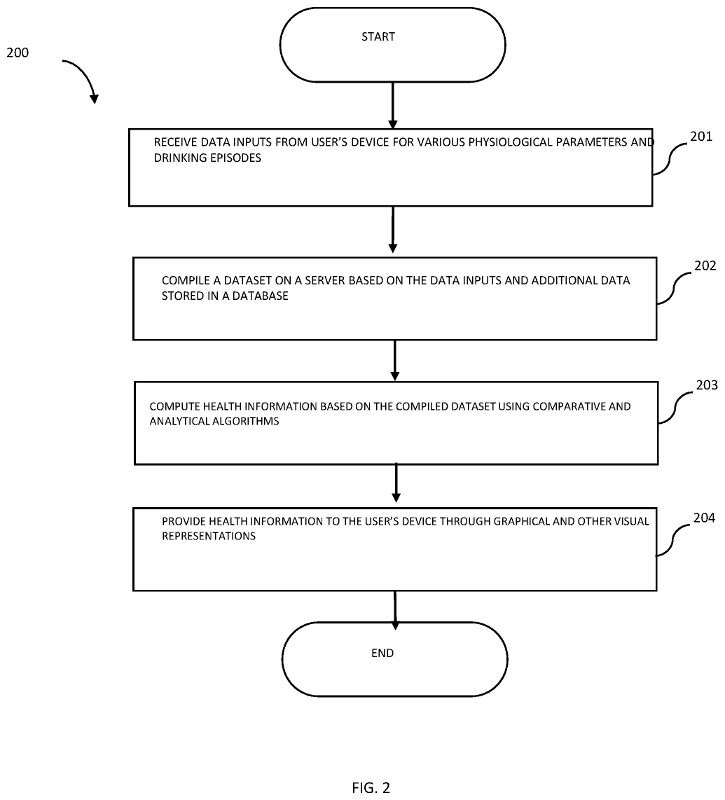

The system 100 of FIG. 1 may be used to implement the various methods presented in FIGS. 2-4 below.

FIG. 2 is a flow diagram illustrating an example method or system 200 for computing and delivering to a user health information related to the user's consumption of alcohol. As in block 201, data inputs from a user's device for various physiological parameters can be received. For example, such information may include the user's weight, height, age, ethnicity, or sex. The data may also comprise, for example, any combination of the following: a user's alcohol ingestion, a user's food ingestion, a user's water ingestion, and a user's time of alcohol ingestion. The data may also comprise the user's movement, genotype, geolocation, gait, and heart rate, or blood oxygenation. As in block 202, the sent data can be compiled into a dataset on a server that includes the sent data and additional data. As in block 203, the dataset on the server can be used by processor to compute health information. As in block 204, the health information can be sent over a network to the user's device and displayed to the user. This process can be repeated. Additional data can be included in the dataset of 202. For example, the data can be the user's mood, exercise, sleep information, or movement. The received data can be manually inputted by the user, or it may be automatically collected. For example, automatic collection may be achieved by APIs into various other software applications such as Apple Health, MyFitness Pal, and other software that may be available.

In an exemplary embodiment, the computed health information of 203 may comprise number of drinks consumed, days per week of alcohol consumed, calories consumed from alcohol consumption, estimation of amount of money spent on alcohol consumed during a period, correlation of mood to drinks, alcohol versus water consumed, estimated blood alcohol concentration, and other information as described in this disclosure. In an exemplary embodiment, the health information provided to the user as in block 204 may be presented graphically, audibly, and may include text, graphs, pictures, and other means of displaying information. In an exemplary embodiment, the information presented to the user as in 204 may be shared with other users of the system. In some embodiments, the shared information may be used by various users to socialize with each other using their devices such as smartphones.

The datasets of 202 can be input to a machine learning model to train the machine learning model to generate predictions of the target metric, such as blood alcohol concentration for example. In one example, a first version of the machine learning model can be trained using the datasets. One or more values in a dataset associated with a prediction driver can be modified to create a modified dataset, and a second version of the machine learning model can be trained using the modified dataset. Performance of the versions of the machine learning model can be compared and a version of the machine learning model can be selected based on the performance of the machine learning model.

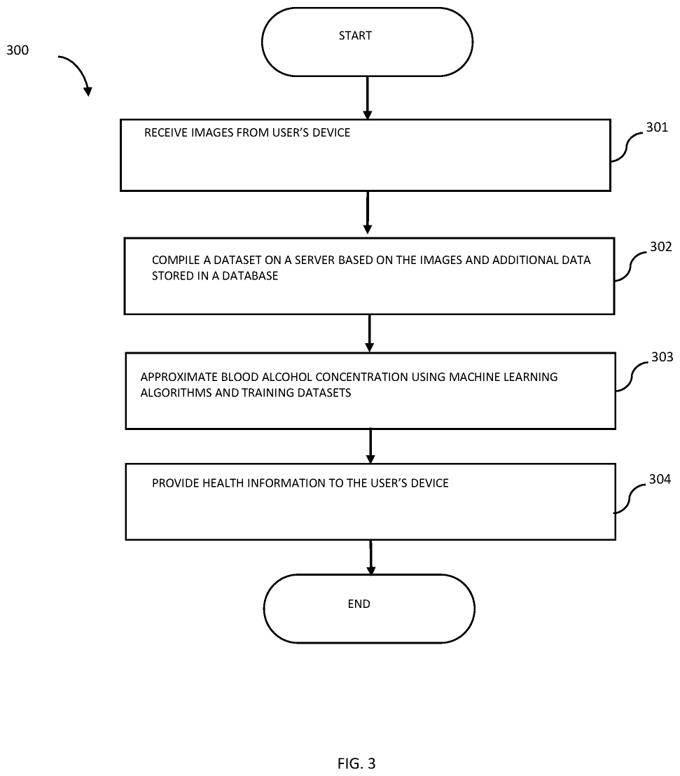

FIG. 3 is a flow diagram illustrating an example method or system for computing and delivering to a user health information related to the user's consumption of alcohol which comprises using the user's images. For example, in block 301, the images may be obtained from the user's smartphone camera. Several images may be taken during a period. The images may be taken during a drinking episode involving alcohol. In an exemplary embodiment, the image may be one or more pictures of the user's face. Several pictures may be taken during a user's drinking episode. The images may be taken along with a breathalyzer reading. In an exemplary embodiment, the additional data of block 302 may comprise, for example, any combination of the following: a user's alcohol ingestion, a user's food ingestion, a user's water ingestion, and a user's time of alcohol ingestion. It may also comprise blood alcohol concentration taken from a breathalyzer.

The computing health information of 303 may comprise estimating blood alcohol concentration based on one or more images of the user's face. Images of the user's face may be used to train a machine learning algorithm to determine blood alcohol concentration. For example, a user may take several pictures of their face starting without having consumed alcohol, while drinking alcohol, and following alcohol consumption. Breathalyzer measurements of blood alcohol concentrations can be taken along with pictures taken of the user's face. The breathalyzer blood alcohol concentration measurements may be used to train a machine learning model via supervised learning using a plurality of users' data, wherein substantially each of the users' data comprises pictures accompanied with their breathalyzer readings substantially correlated in time with the facial pictures. In some exemplary embodiments, the machine learning model can be a convolutional neural network. In some exemplary embodiments, transfer learning can also be used. In some embodiments, the machine learning model could be a binary classifier of intoxicated versus not intoxicated. In some embodiments, the machine learning model can be trained so that the system 300 can estimate various levels of blood alcohol concentration from one or more of the user's pictures.

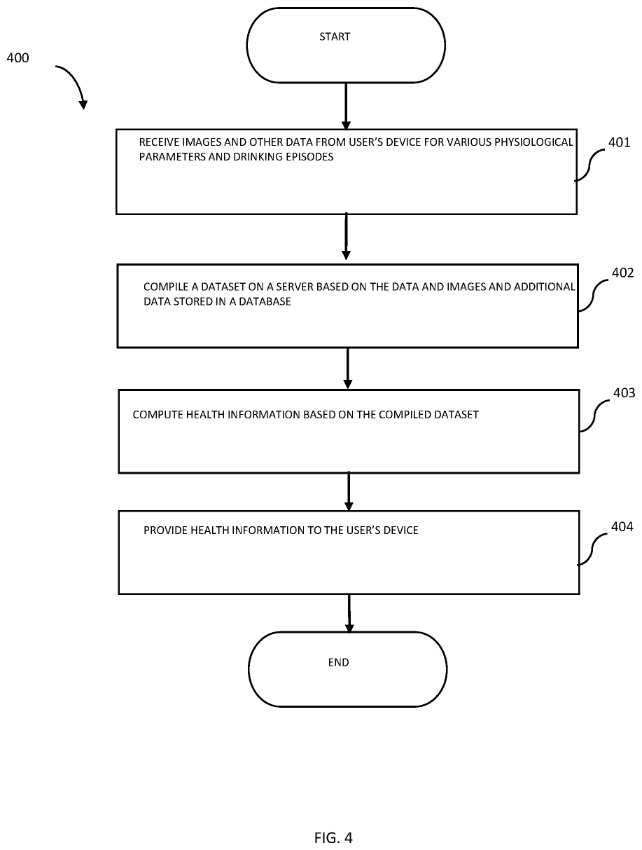

FIG. 4 is a flow diagram illustrating an example method or system for computing and delivering to a user health information related to the user's consumption of alcohol which comprises using the user's images and other information related to the user's consumption of alcohol. For example, in block 401, the images may be obtained from the user's smartphone camera. Several images may be taken during a period. The images may be taken during a drinking episode involving alcohol. In an exemplary embodiment, the image may be one or more pictures of the user's face. Several pictures may be taken during a user's drinking episode. The images may be taken along with a breathalyzer reading. In an exemplary embodiment, the additional data of block 402 may comprise, for example, any combination of the following: a user's alcohol ingestion, a user's food ingestion, a user's water ingestion, and a user's time of alcohol ingestion. It may also comprise blood alcohol concentration taken from a breathalyzer.

The computing health information of 403 may comprise estimating blood alcohol concentration based on one or more images of the user's face along with additional data obtained from the user of 402. Images of the user's face may be used to train a machine learning algorithm to determine blood alcohol concentration. For example, a user may take several pictures of his or her face starting without having consumed alcohol, while drinking alcohol, and following alcohol consumption. Breathalyzer measurements of blood alcohol concentrations can be taken along with pictures taken of the user's face. The breathalyzer blood alcohol concentration measurements may be used to train a machine learning model via supervised learning using a plurality of users' data, wherein substantially each of the users' data comprises pictures accompanied with their breathalyzer readings substantially correlated in time with the facial pictures and additional in addition to other data from 402. In some exemplary embodiments, the machine learning model can be a convolutional neural network. In some exemplary embodiments, transfer learning can also be used. In some embodiments, the machine learning model could be a binary classifier of intoxicated versus not intoxicated. In some embodiments, the machine learning model can be trained so that the system 400 can estimate various levels of blood alcohol concentration from one or more of the user's pictures.

The systems and methods of the exemplary embodiments of FIGS. 1-4, and other embodiments of the present disclosure, may compute various health information and estimate blood alcohol concentration using various parameters related to alcohol absorption and metabolism, as further discussed in the exemplary embodiments below:

### Alcohol Absorption/Metabolism

After alcohol is ingested, it enters the stomach, where a small amount can be absorbed into the bloodstream. Alcohol continues to the small intestine, where a majority of absorption occurs. Once absorbed, alcohol moves across epithelial cells present in the stomach and small intestine, through an interstitial space, and into capillaries in the bloodstream. From circulation, alcohol is carried to the liver via the portal vein, where it is acted upon by enzymes and metabolized. There are a variety of factors that influence the absorption of alcohol. Alcohol absorption in the duodenum and jejunum of the small intestine is more rapid than that which occurs in the stomach. Thus, the gastric emptying rate of the stomach into the small intestine is an important factor in the absorption rate of orally administered alcohol. Alcohol also passes across biological membranes via passive diffusion down a concentration gradient. Therefore, a higher concentration of alcohol will result in a greater concentration gradient and more rapid absorption. Consequently, the presence of food in the stomach will slow gastric emptying through the release of secretins and other hormones, ultimately reducing the absorption rate of alcohol. Thus, the blood alcohol concentration (BAC) may be determined, in part, by the presence or absence of food in the stomach and the amount of alcohol ingested, which affect gastric emptying and rate of oxidation, respectively.

Before entering systemic circulation, some of the alcohol ingested may be metabolized in the stomach by the enzyme alcohol dehydrogenase (ADH) and its isoforms. This is referred to as first pass metabolism and can modulate the bioavailability and toxicity of alcohol. In a fasted state, alcohol rapidly passes from the stomach into the duodenum of the small intestine, minimizing first pass metabolism and leading to higher BAC levels than would be observed in the fed state. While gastric emptying impacts first pass metabolism, the greater levels of metabolizing enzymes in the liver compared to the stomach indicate the liver's major role in alcohol metabolism. In the liver, ADH, and to a lesser extent, cytochrome P450-dependent ethanol-oxidizing system (CYP2E1) are the major enzymatic systems responsible for oxidizing ethanol. The oxidative pathway entails the conversion of ethanol to acetaldehyde by ADH or CYP2E1, and from acetaldehyde to acetate by acetaldehyde dehydrogenase 2 (ALDH2). There are a variety of factors that modify the metabolism and elimination rate of alcohol. Ethanol follows a concentration-dependent rate of elimination; higher BAC levels result in higher rates of alcohol elimination. Age, sex, genetics, body composition, fasted vs. fed state, enzyme levels, and drug interactions also create variations in alcohol metabolism and elimination, and thus BAC levels as a result, as further discussed below.

### Age

There are multiple age-related physiological and anatomical changes, especially with regards to the liver, that are responsible for differing effects of alcohol in older individuals. From a microscopic standpoint, a reduction in the number of hepatocytes is seen in the elderly. Studies have also noted that volume and blood flow to the liver is reduced in the elderly. Collectively, these factors result in an adverse effect on ethanol elimination, resulting in increased BAC and a prolonged effect of alcohol as one ages. The activity of enzymes involved in the metabolism of ethanol are also affected by increasing age. ADH activity in the gastric mucosa of the stomach has been found to vary with age. Before age 60, women demonstrate lower ADH activity than do men, but between the ages of 50 and 60, male ADH activity drops towards the levels noted in females. This reduced ADH activity can contribute to elevated BAC levels in the elderly. Likewise, the enzyme system CYP2E1 has also been seen in studies to demonstrate an age-dependent reduction in activity. Water distribution volume decreases with age, which is of significance because ethanol is a polar substance distributed in the water space. Consequently, BAC levels are significantly higher in those of advanced age. Other factors associated with increased age may include greater prevalence of comorbidities such as diabetes and coronary artery disease (CAD) as well as greater degree of arterial calcification, artherosclerosis, and other vascular etiologies; all of which could impact ethanol absorption into the bloodstream and its transport to the liver.

### Sex

Early studies have been able to confirm that when given an equal amount of alcohol orally, women develop higher BAC levels than do men, despite a faster rate of ethanol elimination. Recent research has been able to confirm this finding that the ethanol oxidation rate is faster in women than in men. This indicates that the difference in alcohol metabolism, and consequently BAC levels, is not solely a function of the liver, but rather involves a myriad of other factors. One of these factors has been found to be differences in enzymatic activity between males and females. Specifically, it appears that x-ADH, a specific isoform of the ADH enzyme present in the stomach, has lower activity in females than in males. This leads to a lower first pass metabolism, increasing ethanol bioavailability, and thus BAC, in women as compared to men. Additionally, volume of distribution has also been identified as a factor in the sex differences in alcohol metabolism. However, when elimination rate is calculated in relation to liver mass, a non-significant difference is found. This indicates that volume of distribution in terms of total body water is a factor in alcohol metabolism. Females typically have a decreased volume of ethanol distribution when compared to men, contributing to higher BAC levels as a result.

### Genetics

Alcohol metabolism is greatly variable from individual to individual and is thought to be a function of genes, and thus alcohol metabolizing enzymes, expressed. ADH and ALDH2 are the primary enzymes involved in alcohol metabolism. Both occur in multiple forms that are encoded by varying genes; there are alleles, or variants, of some genes that encode enzymes with differing characteristics, impacts on metabolism, and that have varying distributions across ethnicities. To date, researchers have primarily studied coding variants of the ADH1B, ADH1C, and ALDH2 genes and the associated altered properties of these enzymes. For example, certain ADH1B and ADH1C enzyme variations result in more rapid conversion of ethanol to acetaldehyde. Specifically, ADH1B*2 and ADH1B*3 alleles are associated with higher oxidative capacity, or more rapid conversion of ethanol to acetaldehyde. In terms of ethnic distribution, ADH1B*1 is found predominantly in Black and Caucasian populations, with ADH1B*2 being higher in frequency in Japanese and Chinese populations. Individuals with Jewish heritage carrying the ADH1B*2 allele show slightly higher alcohol elimination rates compared to those with ADH1B*1. African Americans with the ADH1B*3 allele likewise metabolize ethanol faster than those with alternative alleles. Perhaps the most well-known phenomenon with regards to genetic differences and alcohol metabolism is that of “flushing” in individuals of Eastern Asian descent. A significant polymorphism of the ALDH2 gene results in essentially inactive variants of ALDH2*1 and ALDH2*1. This variation is present in about 50 percent of Han Chinese, Taiwanese, and Japanese populations and accounts for a significant lack of acetaldehyde metabolism. It is this buildup of acetaldehyde in the body that causes the stereotypical facial “flushing” in individuals with this genetic variation.

### Empty vs Full Stomach

Generally, the rate of alcohol oxidation is capped due to the enzymatic activity levels of ADH. In addition to acting in the liver, specific isoforms of ADH act upon alcohol in the stomach, working to metabolize the alcohol before it can be passed to the small intestine and subsequently absorbed into the bloodstream. This phenomenon is termed first-pass metabolism. Thus, factors that decrease gastric emptying into the small intestines, particularly the duodenum and jejunum, serve to increase the time of contact between ADH and the alcohol. In a fed state, not only are the levels of available ADH enzymes increased, but rate of gastric emptying is also reduced, resulting in a greater number of ADH enzymes available to process ethanol molecules and a longer contact time between the two. More specifically, meals high in protein and carbohydrates serve to stimulate secretin and cholecystokinin release, further slowing gastric emptying. A fed state will also serve to increase blood flow to the liver and replenish the supply of reducing molecules required for the conversion of ethanol to acetaldehyde in the mitochondria, thereby accelerating ethanol metabolism.

### Drug-Alcohol Interactions

Just as alcohol can work to alter the pharmacokinetics of prescription drugs, drugs can work to impact pharmacodynamics of ethanol through alterations to the first-pass metabolism and metabolic enzyme activity levels. As mentioned above, factors that alter the activity or amount of ADH, such as H2 receptor blockers and Aspirin (ASA) will result in modified ethanol metabolism. In addition to ADH, CYP2E1 accounts for 10% of ethanol metabolism at lower BAC levels, however this value increases as BAC increases due to the decreased degradation of the CYP2E1 complex—a protective effect to remove xenobiotic materials from the body. Thus, chronic alcohol use results in a greater concentration of CYP2E1 and quicker ethanol metabolism. Similarly, medications that work to induce or inhibit the cytochrome p450 complex can bring about modified elimination rates. While drugs that inhibit ADH and CYP2E1 hold the most theoretical clinical significance, other drug targets such as catalase or acetyladehyde dehydrogenase may also play a role in altering ethanol metabolism.

### BMI and Total Body Water

Unlike other nutrients ethanol is not stored as glycogen in the liver, within fat cells or in skeletal muscle, as is the case with carbohydrates, fats, and proteins respectively. Instead, ethanol remains in the body water until it can be processed by the liver and eliminated. Essentially, an individual's total body water works (TBW) to dilute the ethanol content throughout the body. Because of this, differences in BMI result in different reservoirs of body water for ethanol to reside in, which results in individuals of a higher BMI having a lower BAC than someone of a lower BMI, despite consuming the same amount of alcohol. Moreover, an individual's body fat percentage can play a role in ethanol metabolism and BAC readings. Because fat inherently holds less water than muscle, those with a higher body fat percentage will have a less water and consequently a higher BAC than an individual with a lower body fat percentage.

### Exemplary Machine Learning Algorithms

One exemplary machine-learning algorithm used to generate first machine-learning model may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

One exemplary machine-learning algorithms may include unsupervised processes; unsupervised processes may, as a non-limiting example, be executed by an unsupervised learning module executing on server and/or on another computing device in communication with server, which may include any hardware or software module as described as described herein. An unsupervised machine-learning process, as used herein, is a process that derives inferences in datasets without regard to labels; as a result, an unsupervised machine-learning process may be free to discover any structure, relationship, and/or correlation provided in the data. For instance, and without limitation, expert learner and/or server may perform an unsupervised machine learning process on training set, which may cluster data of training set according to detected relationships between elements of the training set, including without limitation correlations of behavior modifications to each other and correlations of expert qualities and/or categories of experts to each other; such relations may then be combined with supervised machine learning results to add new criteria for expert learner to apply in relating at least a request for a behavior modification to an expert quality. As a non-limiting, illustrative example, an unsupervised process may determine that a first element of behavior modification data closely with a second element of behavior modification data, where the first element has been linked via supervised learning processes to a given expert quality, but the second has not; for instance, the second element may not have been defined as an input for the supervised learning process, or may pertain to a domain outside of a domain limitation for the supervised learning process. Continuing the example, a close correlation between first element of behavior modification and second element of behavior modification may indicate that the second element is also a good predictor for the expert quality; second element may be included in a new supervised process to derive a relationship or may be used as a synonym or proxy for the first behavior modification.

The systems and methods of the exemplary embodiments of FIGS. 1-4, and other embodiments of the present disclosure, may provide various combinations of health information, comprising, for example:

Pie charts comprising: alcohol vs water percentage split (over a specific day, over a 7 previous day period, over a previous 30 day period); Drink Type Distribution;

Graphs, comprising: Bar graphs, where each bar is a log of calories for a drink (using NIH calorie conversion), over 24 hours, a number at the bottom shows total calories; bar graphs over the past 7 days (each bar is a day); “standard” drinks per day; bar graphs over the past 7 days for water (oz), bar graphs for past 7 days and number of logs (encourage more logging on xyz day); and

Additional data comprising user feedback such as: on drink-free days, you log a positive mood x number of times more than on drinking days; you have logged xyz drink free days in a row; Camel: Happy hump day! On average, you drink xyz glasses on Wednesdays; Symbol of beverage type: on x days, you prefer y drink type; you prefer soft beverages to hard liquor; confetti icon, welcome back to Ethos! Start with a log; Most common food type consumed within 4 hours of drinking; Drinking water is suggested; Haven't logged any water today—log suggested; We noticed that you have been drinking x % of [drink 1] and y % of [drink 2]→click to check graph; we noticed that this [time interval] you have been drinking x % of water vs y % of alcohol; we noticed that you have [total] calories from your alcohol consumption; we suggest slowing down drinking (calculating done internally based on reported weight/sex and drinks over given time, for example).

Additionally, correlations and scatter plot data can be computed and presented to users, comprising for example: correlation between drink type and day; we noticed that when you don't drink alcohol your sleep increases by x %; we noticed that when you don't eat and drink alcohol your sleep decreases by x %; we noticed that when you eat before you drink you mood increases by [measurement]; we noticed that when you eat and drink water before drinking, your stress level decreases by [measurement]; we noticed that you tend to drink x % higher when you are in [value] setting; correlation between number of drinks and day; correlation between mood and number of drinks (given: should be a positive correlation between drinking less and mood); correlation between mood and when drinks are consumed in the day; and correlation between mood and drink/water split.

Another exemplary machine-learning algorithm comprises use of data of passive transdermal alcohol concentration (TAC) as an input to estimate BAC. During the process of alcohol metabolism, 1% of alcohol that metabolizes in the liver is excreted in the form of sweat. Transdermal data may be collected using a transdermal device that measures the concentration of alcohol in sweat. In an exemplary embodiment, this data may be used to train machine learning algorithms to estimate BAC. In some embodiments, TAC may be used along with additional data obtained from users as in blocks 202, 302, and 402 of FIGS. 2-4.

### Example: Mobile Application Trained with Large Language Model

One exemplary embodiment comprises a method and system for utilizing a chatbot, trained with a large language model, to provide users with personalized feedback and health tips about monitoring and reducing their alcohol consumption.

An exemplary user interface of a mobile application is configured to collect a variety of data from users about their alcohol consumption such as the quantity and frequency of their drinks, the types of alcoholic beverages they consume, the context in which they're drinking, as well as some personal details such as their age, weight, and gender. The application may be configured to collect self-reported data and data obtained via API with other applications such as Apple Health, Fitbit, or other apps known to those skilled in the art, about the physical and mental impact of users' alcohol consumption.

The obtained data can then undergo a preprocessing stage by a computer system, which converts the raw data into a form that can be used effectively by a large language model (LLM). This preprocessing stage may comprise several processes.

In an exemplary embodiment, the data is cleaned and standardized, comprising removing any extraneous characters and putting data points such as dates and times in a consistent format. Also, abbreviations in the data may be converted into full words.

The standardized data can be tokenized by the computer system, which breaks down the text into smaller pieces.

This tokenized data can then be formatted into a conversation or a prompt format. The user's alcohol consumption data and its effects can be structured into a coherent narrative that can serve as an input to the LLM.

The LLM can then provide users with prompted or unprompted feedback and health tips about their alcohol consumption.

The user data related to alcohol consumption may include various types of data points, such as, for example: the number of drinks consumed during a period, the type of alcohol consumed, the time and context of drinking personal details that may affect alcohol metabolism (age, weight, gender, etc.), and self-reported impact of drinking (mood changes, impact on work or personal life, etc.).

This data could be entered by the user into the mobile application, which could use various types of user interface elements such as text fields, dropdown menus, sliders, or checkboxes. The application might also provide an easy-to-use logging feature for the user to record each drink as they consume it. Some of the data could also be collected automatically. Some of the data could also be obtained via geolocation, pictures, or videos as described in various embodiments as disclosed.

The exemplary LLM-trained application may comprise a chatbot to interact with the user. The chatbot can answer questions about alcohol consumption, reducing alcohol consumption, the physiological and mental effects of alcohol consumption, and the cost of the alcohol consumption. The chatbot could provide the user with a dynamic regimen based on goals such as reducing alcohol consumption, improved sleep, improved memory, improved mood, saving money, or any combination thereof.

### Example: Blood Alcohol Concentration Estimation Feasibility Study

One exemplary feasibility study examines how the rate of change of alcohol metabolism varies from subject to subject? This exploratory study design examines blood alcohol concentration of fed vs fasted state and obtains data used to train a machine learning algorithm to estimate a user's blood alcohol concentration. Various study parameters comprise the following:
100 study subjects: (50% male, 50% female);
Subject Inclusion: healthy adults between 21 and 32 years old;
Subject Exclusion: on any prescription medications, history of alcoholism, underlying health conditions (Heart, Diabetes, Liver, Kidney, GI surgeries/GERD, ulcers).
Data to be collected and Analyzed: app-based physical parameters comprising: age, sex, height, weight, ethnicity, BMI, total body water, breathalyzer data comprising 10 BrAC readings (current BrAC+time until sober measurements) over the entire study, 10 facial photos per subject directly following breathalyzer recordings, average weekly alcohol intake, past medical history, medications, fitness level (how many minutes of physical exercise do you get a week, diet, last meal intake time and contents, stress level scale of 1-5 (1=no stress whatsoever, 3=approaching deadline, 5=familial death), ethnicity, number of water bottles a day on average, types, timing and volumes of alcohol consumed, various BAC breathalyzer readings following number of drinks. Accompanying the readings may be pictures of the participants in direct lighting taken on the investigator's smartphone.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and/or advantages. These also include embodiments in which the components and/or steps are arranged and/or ordered differently.

Unless otherwise stated, all measurements, values, ratings, positions, magnitudes, sizes, and other specifications that are set forth in this disclosure are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

In this disclosure, the indefinite article “a” and phrases “one or more” and “at least one” are synonymous and mean “at least one”.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A computing system comprising:
at least one processor;
a non-transitory computer-readable medium; and
program instructions stored on the non-transitory computer-readable medium that are executable by the at least one processor to cause the computing system to:
obtain data from a user of the computer system representing at least one physiological parameter of the user, wherein the physiological data comprises at least one image of the user's face;
obtain data from a user of the computer system representing the quantity and type of ethanol consumed by the user;
transmit the data to a database; and
generate an estimation of the user's blood alcohol concentration from the user's data.

2. The computing system of claim 1, wherein the data representing the quantity and type of ethanol consumed is provided to the computer system by the user prior to the user consuming the beverage.

3. The computing system of claim 1, wherein the received data further comprises at least one video comprising of at least one beverage comprising ethanol.

4. The computing system of claim 1, wherein the computer system is configured to obtain data representing the user's blood alcohol concentration from a measurement device.

5. The computing system of claim 4, wherein the measurement device is a breathalyzer.

6. The computing system claim 1, wherein the program comprises a trained machine learning model trained on a dataset comprising a plurality of images of a plurality of users' faces and a plurality of said user's blood alcohol concentration measurements.

7. The computing system of claim 6, wherein for each user from whom images of said user's face and said user's blood alcohol concentration measurements are obtained, each image is obtained substantially contemporaneously with a temporally associated blood alcohol measurement.

8. The computing system of claim 7, wherein the machine learning model comprises a convolutional neural network.

9. The computing system of claim 8, wherein the program processes the received data in the trained machine learning model to generate a predictive output, wherein the predictive output is an estimation of the user's blood alcohol concentration.

10. The computing system of claim 9, wherein the predictive output comprises an estimation of the blood alcohol concentration of the user at the time the program generates the prediction.

11. The computing system of claim 9, wherein the predictive output comprises at least one estimation of what the user's blood alcohol concentration is predicted to be at least fifteen minutes after the program generates the prediction.

12. The computing system of claim 9, wherein the predictive output comprises estimations of what the user's blood alcohol concentration is predicted to be at various intervals from between about fifteen minutes to two hours after the program generates the prediction.

13. The computing system of claim 11, wherein after the user consumes at least one additional alcoholic beverage, the computer system provides at least one new estimation of what the user's blood alcohol concentration is predicted to be at least fifteen minutes after the program generates the prediction.

14. The computing system of claim 1, wherein the physiological data obtained by the computer system comprises the user's height and weight.

15. The computing system of claim 13, wherein the program further obtains at least one of the following data: the user's alcohol ingestion, the user's food ingestion, the user's water ingestion, or the user's time of alcohol ingestion.

16. The computing system of claim 1, wherein the program obtains the following data: the user's height, the user's weight, the user's alcohol ingestion, the user's food ingestion, the user's water ingestion, and the time of the user's alcohol ingestion.

17. A computing system comprising configured to provide personalized feedback and health tips for alcohol consumption, comprising:
at least one processor;
a non-transitory computer-readable medium; and
program instructions stored on the non-transitory computer-readable medium that are executable by the at least one processor to cause the computing system to:
receive user data related to alcohol consumption via a mobile application interface;
preprocess said user data to create input suitable for a large language model;
input the preprocessed data into a trained chatbot, wherein the chatbot is trained using a large language model on a dataset comprising conversations and responses related to alcohol consumption, its effects, and methods for reduction;
generate personalized feedback and health tips based on the user data by the chatbot using the trained large language model; and
provide the personalized feedback and health tips to the user via the mobile application interface.

18. The computing system of claim 17, wherein the received data comprises:
the age of the user;
the weight of the user;
the height of the user;
the gender of the user;
the type of alcoholic beverage consumed by the user; and
the time of consumption of the alcoholic beverage.

19. The computing system of claim 18, wherein the received data further comprises the user's self-reported physical and mental state.
